SEQUENCE LISTING (15 sequences)

SEQ ID NO: 1 - DNA, 80 bp, synthetic construct
  source 1..80: mol_type = other DNA
  misc_difference 21..60: note = randomised region optimised for equal probability of incorporation of a, c, g, or t
tcgcacattccgcttctaccnnnnnnnnnnnnnnnnnnnnnnnnnnnnnnnnnnnnnnnncgtaagtccgtgtgtgcgaa

SEQ ID NO: 2 - DNA, 20 bp, synthetic construct
  source 1..20: mol_type = other DNA
tcgcacattccgcttctacc

SEQ ID NO: 3 - DNA, 20 bp, synthetic construct
  source 1..20: mol_type = other DNA
ttcgcacacacggacttacg

SEQ ID NO: 4 - DNA, 20 bp, synthetic construct
  source 1..20: mol_type = other DNA
ggtagaagcggaatgtgcga

SEQ ID NO: 5 - DNA, 20 bp, synthetic construct
  source 1..20: mol_type = other DNA
ttcgcacacacggacttacg

SEQ ID NO: 6 - DNA, 40 bp, synthetic construct
  source 1..40: mol_type = other DNA
tggacgaggtatctcgtatcattggcacctaaaactccgt

SEQ ID NO: 7 - DNA, 40 bp, synthetic construct
  source 1..40: mol_type = other DNA
acctgctgacattgggtgggtcctgaaccatttttaatca

SEQ ID NO: 8 - DNA, 40 bp, synthetic construct
  source 1..40: mol_type = other DNA
caactatttcagtattctagaaaggtcaaaccttaggagc

SEQ ID NO: 9 - DNA, 40 bp, synthetic construct
  source 1..40: mol_type = other DNA
caaggatcggctcaacttaatattggagggatagtgttgg

SEQ ID NO: 10 - DNA, 80 bp, synthetic construct
  source 1..80: mol_type = other DNA
tcgcacattccgcttctacctatgcccaaatccctcaagtcggccaggatacaccaccgtcgtaagtccgtgtgtgcgaa

SEQ ID NO: 11 - DNA, 80 bp, synthetic construct
  source 1..80: mol_type = other DNA
tcgcacattccgcttctaccacctgctgacattgggtgggtcctgaaccatttttaatcacgtaagtccgtgtgtgcgaa

SEQ ID NO: 12 - DNA, 80 bp, synthetic construct
  source 1..80: mol_type = other DNA
tcgcacattccgcttctacccaactatttcagtattctagaaaggtcaaaccttaggagccgtaagtccgtgtgtgcgaa

SEQ ID NO: 13 - DNA, 80 bp, synthetic construct
  source 1..80: mol_type = other DNA
tcgcacattccgcttctacccaaggatcggctcaacttaatattggagggatagtgttggcgtaagtccgtgtgtgcgaa

SEQ ID NO: 14 - DNA, 53 bp, synthetic construct
  source 1..53: mol_type = other DNA
tcgtcggcagcgtcagatgtgtataagagacagtcgcacattccgcttctacc

SEQ ID NO: 15 - DNA, 54 bp, synthetic construct
  source 1..54: mol_type = other DNA
gtctcgtgggctcggagatgtgtataagagacagttcgcacacacggacttacg

(19) United States
(12) Patent Application Publication
(10) Pub. No.: US 2025/0347705 A1
(43) Pub. Date: Nov. 13, 2025

(54) Epitope-Specific hCG Binding Aptamers and Applications Thereof

(71) Applicant: Rhodes University, Grahamstown (ZA)

(72) Inventors: Janice Leigh Limson, Grahamstown (ZA); Lauren Ferreira, Grahamstown (ZA); Ronen Fogel, Grahamstown (ZA)

(73) Assignee: Rhodes University, Grahamstown (ZA)

(21) Appl. No.: 18/871,107

(22) PCT Filed: Jun. 2, 2023
(86) PCT No.: PCT/IB2023/055684

§ 371 Date: Dec. 2, 2024

Related U.S. Application Data
(60) Provisional application No. 63/348,174, filed on Jun. 2, 2022.

(51) Int. Cl. G01N 33/76; C12N 15/115; G01N 33/543

(52) U.S. Cl. CPC G01N 33/76; C12N 15/115; G01N 33/54326; C12N 2310/16; G01N 2333/59

(57) ABSTRACT

This invention relates to aptamers having selectivity and/or specificity for human chorionic gonadotropin (hCG). Also provided for are a biosensor comprising the aptamers having selectivity and/or specificity for hCG and a method for detecting human chorionic gonadotropin (hCG) in a sample using the aptamers or biosensor of the invention, comprising detecting binding of the aptamers to hCG. The invention also relates to a method of identifying aptamers, from a candidate mixture of nucleic acids, that bind to a distinct site of a target molecule, as opposed to another site on the target molecule.

### CROSS-REFERENCE TO RELATED PATENT APPLICATIONS

This application is a U.S. National Stage Application under 35 U.S.C. § 371 of International Patent Application No. PCT/IB2023/055684 filed Jun. 2, 2023, which claims the benefit of priority of U.S. Patent Application No. 63/348,174 filed Jun. 2, 2022, all of which are incorporated by reference in their entireties. The International Application was published on Dec. 7, 2023, as International Publication No. WO 2023/233368 A2.

### SEQUENCE LISTING

The application contains a sequence listing which has been submitted electronically in XML format and is hereby incorporated by reference in its entirety. Said XML copy, created on Feb. 5, 2025, is named US Application-Replacement Sequence Listing-PA177950_US.xml and is 15,000 bytes in size.

### BACKGROUND OF THE INVENTION

This invention relates to aptamers having selectivity and/or specificity for human chorionic gonadotropin (hGC) and to a biosensor comprising the aptamers having selectivity and/or specificity for hCG. The present invention also relates to a method for detecting human chorionic gonadotropin (hCG) in a sample using the aptamers or biosensor of the invention, comprising detecting binding of the aptamers to hCG. Also provided herein is a method of identifying aptamers, from a candidate mixture of nucleic acids, that bind to a distinct site of a target molecule, as opposed to another site on the target molecule.

Human chorionic gonadotropin (hCG) is a glycoprotein hormone secreted by placental trophoblast cells during pregnancy. As such, it is routinely used as a biomarker to diagnose pregnancy in serum and urine samples. Additional research over the last three decades has revealed a complex biological role and substantial variation in composition of this hormone. hCG not only plays a supportive role in pregnancy but is also recognised as a biomarker for trophoblastic cancers, benign gestational trophoblastic diseases, and as a tumour marker for non-trophoblastic human malignancies.

The primary biological function of hCG has traditionally been viewed as replacing luteinising hormone (LH) in stimulating and maintaining progesterone production during the early stages of pregnancy. hCG is a heterodimer consisting of a and B subunits. The α-subunit is identical to that of other glycoprotein hormones: LH, follicle-stimulating hormone (FSH) and thyroid-stimulating hormone (TSH). The β-subunit, although similar to that of LH, is structurally and functionally unique and dictates the specific biological activity of the hormone.

hGC present in blood and urine samples encompasses both the natively expressed, biologically active, heterodimeric form of this molecule and 14 distinct. These are composed of undissociated β subunits, nicks, truncations, and variable glycosylation isoforms, as well as combinations thereof.

Varying levels of hCG are present during the progression of normal pregnancies. Changes in both the hCG levels and the proportions of the various hCG glycoforms and isoforms occur, from predominantly hyperglycosylated autocrine hCG in the first few weeks following implantation to mostly intact, dimeric hCG hormone in the second and third trimesters. The levels and types of hCG are of diagnostic relevance, for example abnormally high or low levels of hCG or its variants can be informative in determining adverse pregnancy outcomes.

Currently, there is a need to develop rapid diagnostic tests capable of recognising and quantifying all hCG variants in both serum and urine samples. Although the diversity of hCG's molecular structure and their respective biological functions are now more fully appreciated, this has not translated into improved design of reagents or devices for the detection and quantification of the molecule and its variants. Most of the currently available methods to detect and monitor hCG levels are antibody-based sandwiching immunoassays, where one antibody, recognising a particular epitope of the target captures the molecule, and a second, labelled antibody, specific to another epitope, completes the sandwich and reports on target presence. Due to this, the complex heterogeneity of hCG variants in human samples complicates accurate measurement of this analyte, as various epitopes are absent or masked in specific hCG glycoforms and isoforms. This heterogeneity leads to substantial differences in the ability of antibodies used in different immunoassays to detect these variants, leading to significant inaccuracies in hCG detection and quantification.

The analytical specificity for a particular variant depends on the epitope(s) recognised by the antibodies used in any given hCG test. Substantial characterisation has been performed to define the epitope specificity of the most commonly used hCG assays in clinical settings. These studies have identified well-characterised epitopes for pan-specific detection of hCG, as well as epitopes specific to particular variants, where, depending on the application, it can be diagnostically advantageous to measure selected clinically relevant variants. To date, the most reliable, but infrequently used, pan-specific antibody combination relies on two antibodies recognising different regions of the β-subunit core fragment that is conserved across all hCG heterodimer and β-subunit variants.

Most commercial hCG tests rely on a combination of antibodies specific to the hCG-specific C-terminal peptide (CTP) of the β-subunit and two epitopes present on the core fragment of the β-subunit, β₂ and β₈. However, the CTP is cleaved in many hCG degradation variants, including the core fragment isoform (hCGβcf), making these tests inappropriate for use in urine, where hCGβcf is the most abundant hCG variant. Consequently, hCGβcf detection by commercially available hCG tests has been reported to be problematic.

In contrast to existing detection platforms, a diagnostically useful epitope contained within the core fragment, β₁, represents an under-researched opportunity for developing specific immunoassays for hCG. Biorecognition elements specific to this epitope are reported to offer superior specificity, a wide recognition of hCG and its variants, and the absence of cross-reactivity with LH (Berger et al., 2013).

In addition to the above, limitations in conventional hCG-detecting immunoassays, antibody-based diagnostic tools provide high target specificity and affinity but have high production costs and limited shelf lives, due to their thermosensitivity. Alternative affinity reagents to antibodies are DNA- or RNA-based aptamers, which boast several advantages over antibodies, including their affordability, thermal stability, ease of chemical modification and potential for large-scale production. In addition, while achieving epitope-specificity with antibodies remains challenging, a number of strategies have been described to obtain epitope-specific aptamers, which represents a further advantage of these nucleic acid based biorecognition molecules.

### SUMMARY OF THE INVENTION

According to the present invention, there is provided for an aptamer that selectively and/or specifically binds human chorionic gonadotropin (hCG) and a biosensor device for detecting human chorionic gonadotropin (hCG) using said aptamer. Also provided is a method for detecting human chorionic gonadotropin (hCG) in a sample by contacting a sample with an aptamer having selectivity and/or specificity for binding human chorionic gonadotropin (hCG), comprising or consisting of the nucleotide sequence of any one of SEQ ID NOs: 7-9, or a complementary sequence thereof and detecting binding of the aptamer to hCG, wherein binding of the aptamer to hCG indicates the presence of hCG in the sample.

According to a first aspect of the present invention there is provided for an aptamer comprising or consisting of the nucleotide sequence of any one of SEQ ID NOs: 7-9, or a complementary sequence thereof, wherein the aptamer selectively and/or specifically binds human chorionic gonadotropin (hCG). Optionally, the aptamer may have the nucleotide sequence of any one of SEQ ID Nos:11-13, or a complementary sequence thereof.

In a first embodiment of the aptamer of the invention, the aptamer binds the β subunit of hCG, preferably the β₁ epitope of hCG.

According to a second embodiment of the aptamer of the invention, the aptamer may be labelled. For example, the label may be selected from the group consisting of biotin, a fluorescent label, a luminescent label, a radioactive isotope, amine, aryl azides, thiol, a nanoparticle, or an enzymatic label. In a preferred embodiment, the label may be biotin.

According to a second aspect of the present invention, there is provided for a biosensor device for detecting human chorionic gonadotropin (hCG), preferably in a sample, comprising at least one aptamer comprising or consisting of the nucleotide sequence of any one of SEQ ID NOs:7-9, or a complementary sequence thereof, wherein the aptamer selectively and/or specifically binds hCG. Optionally, the aptamer may have the nucleotide sequence of any one of SEQ ID Nos:11-13, or a complementary sequence thereof.

In a first embodiment of the biosensor device, the biosensor device may comprise two aptamers. For example, the biosensor device may comprise a first aptamer comprising or consisting of the nucleotide sequence of SEQ ID NO: 7 or SEQ ID NO:8, or a complementary sequence thereof, and a second aptamer comprising or consisting of the nucleotide sequence of SEQ ID NO:9, or a complementary sequence thereof.

According to a second embodiment of the biosensor device, the first or second aptamer may bind the β subunit of hCG, preferably the β₁ epitope of hCG.

In a third embodiment of the biosensor device, at least one of the first or second aptamer is labelled. For example, the label may be selected from the group consisting of biotin, a fluorescent label, a luminescent label, a radioactive isotope, amine, aryl azides, thiol, a nanoparticle, or an enzymatic label. In a preferred embodiment, the label may be biotin.

According to a third aspect of the present invention there is provided for a method of detecting human chorionic gonadotropin (hCG) in a sample, wherein the method comprises: a) providing an aptamer having selectivity and/or specificity for binding human chorionic gonadotropin (hCG), wherein the aptamer comprises or consists of the nucleotide sequence of any one of SEQ ID NOs: 7-9, or a complementary sequence thereof, optionally the aptamer has the nucleotide sequence of any one of SEQ ID Nos: 11-13, or a complementary sequence thereof; b) contacting the sample with the aptamer; and c) detecting binding of the aptamer to hCG, wherein binding of the aptamer to hCG indicates the presence of hCG in the sample.

In a first embodiment of the method, the method may be a sandwich assay and may further comprise contacting the aptamer bound to the hCG with a second aptamer. For example, the aptamer bound to the hCG may comprise or consist of the nucleotide sequence of SEQ ID NO: 7 or SEQ ID NO:8, or a complementary sequence thereof, and the second aptamer may comprise or consist of the nucleotide sequence of SEQ ID NO:9, or a complementary sequence thereof.

According to a second embodiment of the method, the aptamer and/or the second aptamer binds the B subunit of hCG, preferably the β₁ epitope of hCG.

In a third embodiment of the method, at least one of the aptamers may be labelled. For example, the label may be selected from the group consisting of biotin, a fluorescent label, a luminescent label, a radioactive isotope, amine, aryl azides, thiol, a nanoparticle, or an enzymatic label. In a preferred embodiment, the label may be biotin.

In a fourth embodiment of the method, detecting binding of the aptamer to hCG may be performed using an impedimetric assay, a spectrophotometric assay, a voltammetric assay, a chemiluminescence assay, a flow cytometry assay, a radioactive assay, an immunochromatographic assay, a piezoelectric assay, a colorimetric assay, a fluorescence

Also provided herein are kits of parts comprising the aptamers described herein, together with instructions for performing the methods described.

### BRIEF DESCRIPTION OF THE FIGURES

Non-limiting embodiments of the invention will now be described by way of example only and with reference to the following figures:

FIG. 1: Diagrammatic representation of the selection strategy used to enrich for multiple, site-specific DNA aptamers. Two stages of selection were used to enrich aptamers capable of recognising distinct sites of hCG. In the first five rounds, negative selection occurred against Tris-blocked beads, thereby removing sequences recognising the solid support of the magnetic beads. Positive selection was against the target protein, hCG, with round three using FSH as a counter selection step to specifically enrich for aptamers against the β-subunit. From round six, negative selection pressure changed to hCG, coupled with the β₁-epitope specific antibody, on magnetic beads. Any DNA sequence binding to any other part of the hCG molecule was therefore removed from the pool, and only sequences specific to the β₁ epitope were carried through to the subsequent hCG positive selection round.

FIG. 2: Bead surface characterisation by ELISA style assay at each round of selection, by detecting the α-subunit shared by hCG and FSH. The surface of the beads for positive and negative selection steps, at every round of SELEX, was analysed using an HRP-conjugated rabbit anti-goat secondary antibody to the goat anti-hCG α-subunit specific antibody capable of recognising hCG (in all positive selection rounds), FSH (in counter selection rounds 3 and 7) and 1-specific antibody coupled hCG (in the negative selection in rounds 6-9). 100 μg of beads for the first selection round, or 20 μg for each subsequent round, were tested in triplicate for each condition. TMB colour change was measured kinetically over twenty minutes and rate of change is shown. Error bars show standard deviation for triplicate values. A one-way ANOVA with Tukey's post-hoc test was used to analyse differences between the samples in either the negative (F(8, 18)=37.50, p=0.00001) or positive (F(8, 18)=6.45, p=0.00052) selection steps. Conditions generating TMB signals statistically different to those of round 2 are shown as * for the negative selection steps and † for the positive selection steps.

FIG. 3: Absolute DNA quantity obtained by qPCR for negative and positive selection steps at each round of selection. The dashed line between Rounds 5 and 6 indicates the change in selection pressure towards the β₁-epitope specifically.

FIG. 4: Stored samples of ssDNA from pools entering SELEX (1 pmol) were each incubated with 1 mg of beads (coated using 10 μg of hCG) and allowed to bind for an hour. Input DNA, unbound DNA in the supernatant and DNA bound to the target beads were quantified by qPCR. The binding ratio was expressed as the ratio of the amount of bound ssDNA to the amount of input ssDNA at each round. Single samples were used for each round.

FIG. 5: Amplification curves obtained for different selection rounds analysed by qPCR. ssDNA generated from the exonuclease digestion after each SELEX round were used as the template in these reactions.

FIG. 6: Melting curves obtained for different selection rounds analysed by qPCR. ssDNA generated from the exonuclease digestion after each SELEX round were used as the template in these reactions. obtained for different selection rounds analysed by qPCR. ssDNA generated from the exonuclease digestion after each SELEX round were used as the template in these reactions.

FIG. 7: Frequency of selected sequences in SELEX pools, showing the four selected groups of sequences anticipated from the SELEX procedure in this study. Predicted binding specificities of candidate aptamer sequences identified through selection by next generation sequencing.

FIG. 8: Initial screening of aptamer candidates' ability to bind unimmobilised hCG assessed by electrophoretic mobility shift assays. Representative GelRed stained gel images are shown with or without hCG incubation. The difference in pixel intensity of the DNA bands for each sequence was calculated in the presence and absence of hCG using ImageJ analysis software. These values shown are the calculated as change in percentage of the intensity of the unbound DNA band in the presence of hCG, compared to the band formed in the absence of hCG [†=changes in band intensity in the presence of the target ≥10% are considered significant].

FIG. 9: qPCR-based quantification of aptamer sequences binding to beads coated either with hCG or the control protein, HSA. Assays were performed in triplicate, with all three values shown [†=sequences exhibiting significantly higher quantities of DNA bound to hCG-modified beads, compared to HSA-modified beads (two-tailed t-tests, p≤0.05). *=sequences exhibiting significantly higher binding to hCG-modified beads compared to the Lib_1 control sequence (one-way ANOVA results annotated in figure; sequences identified using subsequent Tukey HSD post-hoc test, p≤0.05)].

FIG. 10: Spectrophotometric ELONA assay of biotinylated aptamer sequences binding to beads coated either with hCG or the control protein, BSA. Following exposure of the aptamers to hCG- or BSA-coated magnetic beads, bound aptamers were subsequently quantified using the streptavidin-HRP and TMB reporter system. Assays were performed in triplicate, with all three values shown [†=sequences exhibiting significantly higher quantities of DNA bound to hCG-modified beads, compared to HSA-modified beads (two-tailed t-tests, p≤0.05). ‡=sequences exhibiting suggestively higher quantities of DNA bound to hCG-modified beads, compared to HSA-modified beads (two-tailed t-tests, p≤0.1). *=sequences exhibiting significantly higher binding to hCG-modified beads compared to “No DNA” control samples (one-way ANOVA results annotated in figure; sequences identified using subsequent Tukey HSD post-hoc test, p≤0.05)].

FIG. 11: Paper-format ELONA screening of aptamer sequences. hCG was covalently attached to the paper surface within each well and exposed to biotinylated sequences. Bound sequences were subsequently quantified using an anti-biotin antibody and subsequent quantification using a secondary antibody-HRP and TMB system. Representative images captured of the colorimetric signal generated for different aptamer sequences tested by indirect paper-based ELONA (top panel). The signal intensity for sample was quantified by ImageJ and is presented as a univariate plot (bottom panel) [*=sequences exhibiting significantly higher binding to hCG-modified wells compared to R4_1 control samples (one-way ANOVA results annotated in figure; sequences identified using subsequent Tukey HSD post-hoc test, p≤0.05)].

FIG. 12: Competitive assays used to elucidate aptamer candidates' target epitope specificity: Panel A) Evaluation of conditions required to capture hCG when using aptamer sequence R4_64: Wells were coated with streptavidin, then modified with biotinylated aptamer sequences and exposed to hCG. hCG captured was quantified using the anti-α subunit antibody, subsequently using a HRP-conjugated secondary antibody and TMB to quantify the presence of the anti-a antibody. Different aptamer conditions were compared to control sequences which lacked any biotin modification (unmodified full-length R4_64) and any capturing DNA (no DNA). Triplicate measurements were generated and shown [*=sequences exhibiting significantly higher binding to hCG-modified beads compared to “No DNA” control samples (one-way ANOVA results annotated in figure; sequences identified using subsequent Tukey HSD post-hoc test, p≤0.05)]. Panel B) Screening of aptamer sequences capable of binding to hCG sites that do not compete with R4_64's binding. Full-length R4_64, folded in the presence of the complementary blocking oligonucleotides, was immobilised onto streptavidin-coated magnetic beads through its attached biotin moiety. Beads were exposed to hCG and subsequently, full-length, oligomer-blocked aptamer candidates. Following DNA elution, aptamers bound to the beads were quantified by SYBR qPCR. Triplicate measurements were made and shown [*=sequences exhibiting significantly higher binding to hCG captured by R4_64, compared to the “None” control samples (one-way ANOVA results annotated in figure; sequences identified using subsequent Tukey HSD post-hoc test, p≤0.05)].

### SEQUENCE LISTING

The nucleic acid and amino acid sequences listed herein and in any accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:
SEQ ID NO:1—nucleotide sequence of the SELEX Library template DNA.
SEQ ID NO:2—nucleotide sequence of the Forward post-SELEX PCR Primer.
SEQ ID NO:3—nucleotide sequence of the Reverse post-SELEX PCR Primer.
SEQ ID NO:4—nucleotide sequence of the Forward SELEX Library Primer.
SEQ ID NO:5—nucleotide sequence of the Reverse SELEX Library Primer.
SEQ ID NO:6—nucleotide sequence of the R4_1 truncated aptamer.
SEQ ID NO:7—nucleotide sequence of the R4_64 truncated aptamer.
SEQ ID NO:8—nucleotide sequence of the R6_5 truncated aptamer.
SEQ ID NO:9—nucleotide sequence of the R5_4 truncated aptamer.
SEQ ID NO:10—nucleotide sequence of the Lib_1 full length aptamer.
SEQ ID NO:11—nucleotide sequence of the R4_64 full length aptamer.
SEQ ID NO:12—nucleotide sequence of the R6_5 full length aptamer.
SEQ ID NO:13—nucleotide sequence of the R5_4 full length aptamer.
SEQ ID NO:14—nucleotide sequence of the Forward NGS Primer.
SEQ ID NO:15—nucleotide sequence of the Reverse NGS Primer.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The inventors of the present invention have employed a SELEX procedure tailored towards the identification of two pools of aptamers, one specific to the β-subunit and, another to the β1 epitope of hCG. This was achieved using a novel selection approach to enrich for sequences specific to distinct regions of the target, within a single SELEX experiment. The use of this unique selection strategy, combined with next-generation sequencing (NGS) analysis, allowed for the identification of predicted site-specific sequences. Competition-based molecular biology assays confirmed the anticipated site specificity of the identified aptamers and verified their combined use in a sandwich format. In one embodiment, the aptamers identified herein, designated as R4_64 and R6_5, recognise two distinct sites of the hCG molecule-the β-subunit and the 1-epitope, respectively, which are suitable for use as hCG-specific biorecognition agents for monitoring human pregnancy.

While previous SELEX strategies have been described to isolate aptamers against a particular region of a target, none reported have used antibody-blocked target molecules as a negative selection pressure during SELEX to direct binding to a specific epitope, as was used here. Thus, the present invention also encompasses a new SELEX approach.

The inventors of the present invention describe a novel predetermined epitope-targeted selection strategy for identifying aptamers recognising two distinct sites of the hCG molecule—the β-subunit and the β₁-epitope. This was achieved by iteratively exposing the DNA pool to magnetic beads functionalised with target molecules (positive selection) or non-target molecules (negative and counter selection). While previous SELEX strategies have been described to isolate aptamers against a particular region of a target, none reported have used antibody-blocked target molecules as a negative selection pressure during SELEX to direct binding to a specific epitope, as is described herein. This strategy offers several advantages over other methods, including the need for only a single SELEX experiment to enrich for different pools of aptamers capable of binding discrete sites on the target. This strategy could prove useful for generating aptamer pairs for use in combination in sandwiching assays, similar to antibody-based sensor formats. Additionally, the change of selection pressure within the selection process, analysed by NGS, allowed for discrimination of suspected amplifier artefact sequences and genuine target binding sequences.

Also described herein, the inventors have shown using competition assays that R4_64 and R6_5 bind to an overlapping site on the target, likely the β₁-epitope, which is not shared by R5_4. Importantly, the binding site specificity of R4_64 and R5_4 allows these aptamers to simultaneously capture and report on the target, indicating their successful use in a sandwich format. These sequences are thus useful in subsequent applications as hCG-specific biorecognition agents. The use of these aptamers in a sandwich-style diagnostic test would ensure recognition of the hCGβcf specifically and enable its detection in urine samples, which is currently not possible.

The terms “nucleic acid” or “nucleic acid molecule” encompass both ribonucelotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

As used herein, the terms “oligonucleotide” and “polynucleotide” both refer to DNA or RNA fragments comprising one or more nucleotides, including naturally occurring or non-naturally occurring nucleotides, or nucleotide analogs or derivatives.

The term “aptamer” refers to a single stranded nucleotide sequence that specifically binds to a particular target molecule. The nucleotide sequence is preferably a DNA sequence, although RNA or other amplifiable nucleic acid based polymers, including naturally occurring or non-naturally occurring nucleotides, or nucleotide analogs or derivatives, can be used. The distinct sequences of the aptamers of the present invention determine the folding of the oligonucleotide molecule into a unique conformational structure. Preferably, an aptamer is a degenerate sequence of about 15-120 nucleotides bases, more preferably of about 30-60 nucleotide bases, in length. The aptamers of the present invention may be flanked by fixed sequences. Those of skill in the art will understand that the sequence of the aptamer may be varied without substantially affecting binding of the target molecule to the aptamer. Thus, the term also encompasses aptamers that are substantially identical to the aptamers disclosed herein.

The term “sample” refers to a sample isolated or collected from an environmental or biological source and is located ex vivo. Preferably, the sample is from a biological source, more preferably a blood or fluid sample, such as a urine sample.

The term “isolated”, is used herein and means having been removed from its natural environment.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more substitutions, deletions, or insertions that do not destroy or substantially reduce the binding affinity of the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2XSSC), and a second wash for 10 min at 65° C. in Wash Buffer B (0.1% SDS; 0.5% SSC).

The term “SELEX” as used herein refers to any systematic and iterative technique for the selective enrichment of aptamers by exponential amplification and molecular evolution.

The term “target molecule” or “target” refers to any molecule capable of forming a complex with an oligonucleotide, including, but not limited to, small organic compounds such as drugs, dyes, metabolites, cofactors, transition state analogs, and toxins. Preferably, the target molecule is human chorionic gonadotropin, more preferably the β-subunit of human chorionic gonadotropin, most preferably the β₁ epitope thereof.

The terms “label” and “detectable label” interchangeably refer to a composition detectable by spectroscopic, photochemical, biochemical, immunochemical, electrochemical, chemical, or other physical means. Useful labels include fluorescent dyes (fluorophores), fluorescent quenchers, luminescent agents, electron-dense reagents, enzymes (e.g., as commonly used in an ELISA), biotin, digoxigenin, ³²P and other radioisotopes, gold nanoparticles (AuNPs), haptens, proteins, nucleic acids, or other substances which may be made detectable, e.g., by incorporating a label into an oligonucleotide specifically reactive with a target molecule. The term includes combinations of single labelling agents, e.g., a combination of labels that provides a unique detectable signature.

In some embodiments, the aptamers or aptamer compositions according to the invention may be provided in a kit, together with instructions for use. In other embodiments, the aptamers or aptamer compositions of the present invention may be integrated into an electrochemical impedance spectroscopy biosensing platform for use as a “biosensor” or “aptasensor”.

The following examples are offered by way of illustration and not by way of limitation.

### EXAMPLE 1

### Reagents and Statistical Analysis

### Oligonucleotides for SELEX and Binding Studies

All oligonucleotides were sourced from Integrated DNA Technologies (WhiteSci). For SELEX, a commonly used 80 nt initial ssDNA library comprised of a 40-mer random region flanked on either side by 20-mer primer binding sequences was used. The library was composed of the general sequence: 5′-TCGCACATTCCGCTTCTACC(N₄₀)CGTAAGTCCGTGTGTGCGAA-3′ (SEQ ID NO:1). The randomised regions were prepared combinatorially by mixing A: C: G: T nucleotide bases at molar ratios of 3:3:2:2.4, to optimise equal probability of incorporation of each nucleotide.

Two sets of oligonucleotides were used in combination with the library in this study. The first, a set of primer pairs (Fwd: 5′-TCGCACATTCCGCTTCTACC-3′ (SEQ ID NO:2) and Rv: 5′-TTCGCACACACGGACTTACG-3′ (SEQ ID NO:3)) was used to conduct PCR post-SELEX. The reverse primer was sourced with a 5′phosphorylation modification, to enhance formation of single-stranded DNA during exonuclease digestion after amplification. The second set of synthesised oligonucleotides (Fwd: 5′-GGTAGAAGCGGAATGTGCGA-3′ (SEQ ID NO:4) and R: 5′-TTCGCACACACGGACTTACG-3′ (SEQ ID NO:5)) were designed to complement primer binding sites on the library and was used during SELEX to complement these regions.

Aptamer candidate sequences identified through SELEX in these studies as well as control sequences were sourced from Integrated DNA Technologies (IDT) for empirical testing in validation studies. Table 1 below presents their sequences and designations. Both truncated sequences (lacking primer-binding sites) and full-length sequences were sourced. Truncated sequences, modified on the 5′ end with a biotin moiety, were used as an initial screen of target-binding ability by ELONA methods. Screening of aptamer candidates by qPCR using the full-length DNA sequence was also conducted.

TABLE 1
Aptamer sequences, and their modifications, used in this study.

| | Truncated | | Full-length | |
|---|---|---|---|---|
| | sequence | | sequence | |
| Name | (5′ → 3′) | Modification | (5′ → 3′) | Modification |
| Lib_1 | — | — | TCGCACATTCCGCTTCTACCTA | — |
| | | | TGCCCAAATCCCTCAAGTCGGC | |
| | | | CAGGATACACCACCGTCGTAAG | |
| | | | TCCGTGTGTGCGAA | |
| | | | (SEQ ID NO: 10) | |
| R4_1 | TGGACGAGGTATCTCG | 5′ biotin | — | — |
| | TATCATTGGCACCTAA | | | |
| | AACTCCGT | | | |
| | (SEQ ID NO: 6) | | | |
| R4_64 | ACCTGCTGACATTGGG | 5′ biotin | TCGCACATTCCGCTTCTACCAC | 5′ biotin |
| | TGGGTCCTGAACCATT | 3′ biotin | CTGCTGACATTGGGTGGGTCCT | |
| | TTTAATCA | | GAACCATTTTTAATCACGTAAG | |
| | (SEQ ID NO: 7) | | TCCGTGTGTGCGAA | |
| | | | (SEQ ID NO: 11) | |
| R6_5 | CAACTATTTCAGTATT | 5′ biotin | TCGCACATTCCGCTTCTACCCA | — |
| | CTAGAAAGGTCAAACC | | ACTATTTCAGTATTCTAGAAAG | |
| | TTAGGAGC | | GTCAAACCTTAGGAGCCGTAAG | |
| | (SEQ ID NO: 8) | | TCCGTGTGTGCGAA | |
| | | | (SEQ ID NO: 12) | |
| R5_4 | CAAGGATCGGCTCAAC | 5′ biotin | TCGCACATTCCGCTTCTACCCA | — |
| | TTAATATTGGAGGGAT | | AGGATCGGCTCAACTTAATATT | |
| | AGTGTTGG | | GGAGGGATAGTGTTGGCGTAA | |
| | (SEQ ID NO: 9) | | GTCCGTGTGTGCGAA | |
| | | | (SEQ ID NO: 13) | |

Primer binding sites for individual sequences are underlined.

### Proteins and Antibodies

hCG (≥50%, isolated from human urine, ab126652) was purchased from Abcam, while FSH (7000 I.U./mg, F4021) and human serum albumin (HSA) (99%, A3782) were purchased from Sigma. All proteins were received as lyophilised powders and resuspended in 0.15 M HEPES, adjusted to pH 7.0, to stock concentrations of 1 mg/ml (hCG), 0.25 mg/ml (FSH) and 0.25 mg/ml (HSA), respectively.

Monoclonal mouse anti-hCG beta1 epitope antibody (1 mg/ml, ab11388, Abcam), hereafter referred to as the hCG β₁-epitope specific antibody, was used as a primary antibody to hCG, and detected with polyclonal goat anti-mouse IgG, conjugated with horseradish peroxidase (HRP) (0.1 mg/ml, HAF007, R&D Systems), as the secondary antibody. Polyclonal goat anti-hCG alpha antibody (2.11 mg/ml, ab20712, Abcam), hereafter referred to as the α-subunit-specific antibody, was used as a primary antibody to hCG and FSH. The α-subunit antibody was detected with polyclonal rabbit anti-goat IgG, conjugated with HRP (2 mg/ml, ab6741, Abcam), as the secondary antibody. HRP presence was detected using 1-Step UltraTMB-ELISA (ThermoFisher Scientific) for magnetic bead assays and KPL TMB membrane peroxidase substrate system kit (SeraCare) on paper (TMB=3,3′,5,5′-tetramethylbenzidine).

DynaBead™ epoxy-functionalised M-270 superparamagnetic beads (ThermoFisher Scientific) were used as a solid support for protein coupling in the SELEX experiment.

### Statistical Analysis

Unless otherwise stated, tests requiring repeated measures were performed in, at least, triplicate samples i.e. n≥3. For the univariate plots, means are shown as solid lines, while medians are dashed. Uncertainties in text (x±y) represent means and standard deviation. Statistical differences between two conditions were evaluated using the two-tailed, equal-variance Student's t-test function in Microsoft Excel. One-way ANOVAs, with Tukey HSD post hoc tests, were used for determining and comparing significant differences in means of more than two samples, using Statistica® v13 software. Statistical significance (a) was assigned at 0.05. Variance weighted least squares non-linear regression was fitted to the kinetic binding data using Statistica® v13 software.

### EXAMPLE 2

### SELEX

### Targeted SELEX Strategy to Generate Aptamers Specific to Different Sites of the Target Molecule, hCG

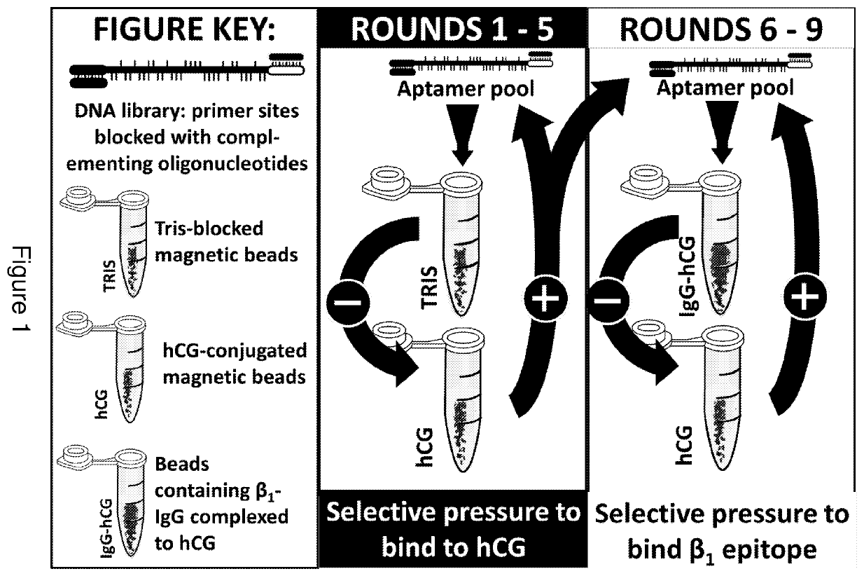

Nine rounds of a novel, epitope-targeted SELEX strategy were completed to enrich for DNA aptamers capable of binding to distinct sites of the hCG target molecule (FIG. 1). For the first five rounds, initial negative selection against Tris-blocked magnetic beads was followed by positive selection against hCG-functionalised magnetic beads.

SELEX proceeded by incubating ssDNA library pools with magnetic beads surfaced with the target proteins. Exposure of the DNA pools to the proteins occurred in a synthetic urine matrix at room temperature. This was formulated similarly to that reported by Chutipongtanate and Thongboonkerd (2010): 200 mM urea, 1 mM uric acid, 3.43 mM creatine, 0.1 mM sodium oxalate, 5 mM sodium citrate, 54.2 mM sodium chloride, 30.2 mM potassium chloride, 15 mM ammonium chloride, 3 mM calcium chloride, 2 mM magnesium sulphate, 2 mM sodium bicarbonate, 9.1 mM sodium sulphate, 3.6 mM sodium dihydrogen phosphate, and 0.4 mM disodium hydrogen phosphate, but supplemented with 2.8 nM glucose and 17 nM HSA and freshly prepared before each selection round. After incubation with the beads, bead-binding or non-binding fractions of ssDNA were isolated and selectively amplified for the next round of selection.

Selection occurred in two phases to identify aptamers capable of binding to the β-subunit as a whole and also the β₁ epitope specifically (FIG. 1). The various counter/negative and positive selection steps used, as well as the stringency applied, are summarised in Table 2. Using this strategy, negative, counter and positive selection were determined by controlling the protein modification present on the magnetic beads. All rounds of SELEX employed the same positive selection pressure-hCG-functionalised magnetic beads. For both negative and counter selections, the unbound sequences remaining in the supernatant were used in the second, positive selection step.

Table 2: SELEX strategy used to select site-specific hCG aptamers. Increasing stringency was applied as the process progressed. Asterisks indicate selection rounds analysed by next-generation sequencing.

| | | | | | | | | | | Wash |
|---|---|---|---|---|---|---|---|---|---|---|
| | | | | | | | | | | regimen |
| | | | | | | | | | | of beads |
| | Library | Negative selection | | | | Positive selection | | | | before |
| SELEX | DNA | Bead | Protein | Beads | Incubation | Bead | Protein | Beads | Incubation | DNA |
| round | (pmol) | coating | (μg) | (mg) | time (min) | coating | (μg) | (mg) | time (min) | elution |

Phase 1: Aptamer pools enriched towards hCG protein

| | | | | | | | | | | |
|---|---|---|---|---|---|---|---|---|---|---|
| 1* | 1000 | Tris- | 0 | 5 | 60 | hCG | 70 | 5 | 60 | 3 × artificial |
| | | blocked | | | | | | | | urine |
| 2 | 200 | Tris- | 0 | 1 | 60 | hCG | 14 | 1 | 60 | 5 × artificial |
| | | blocked | | | | | | | | urine with |
| | | | | | | | | | | 0.005% v/v |
| | | | | | | | | | | TWEEN 20 |
| 3 | 200 | Tris- | 0 | 1 | 60 | FSH S_{N} | 14 | 1 | 60 | 5 × artificial |
| | | blocked | | | | | | | | urine with |
| | | | | | | | | | | 0.005% v/v |
| | | | | | | | | | | TWEEN 20 |
| 4* | 200 | Tris- | 0 | 1 | 60 | hCG | 10 | 1 | 60 | 5 × artificial |
| | | blocked | | | | | | | | urine with |
| | | | | | | | | | | 0.005% v/v |
| | | | | | | | | | | TWEEN 20 |
| 5* | 20 | Tris- | 0 | 1 | 60 | hCG | 10 | 1 | 60 | 5 × artificial |
| | | blocked | | | | | | | | urine with |
| | | | | | | | | | | 0.005% v/v |
| | | | | | | | | | | TWEEN 20 |

Phase 2: Aptamer pools enriched towards β₁ epitope of the hCG protein

| | | | | | | | | | | |
|---|---|---|---|---|---|---|---|---|---|---|
| 6* | 20 | Ab- | 10 Ab + | 1 | 75 | hCG | 10 | 1 | 45 | 6 × artificial |
| | | masked | 10 hCG | | | | | | | urine with |
| | | hCG | | | | | | | | 0.005% v/v |
| | | | | | | | | | | TWEEN 20 |
| 7 | 20 | Ab- | 10 Ab + | 1 | 75 | FSH S_{N} | 10 | 1 | 45 | 6 × artificial |
| | | masked | 10 hCG | | | | | | | urine with |
| | | hCG | | | | | | | | 0.005% v/v |
| | | | | | | | | | | TWEEN 20 |
| 8* | 20 | Ab- | 10 Ab + | 1 | 75 | hCG | 10 | 1 | 45 | 6 × artificial |
| | | masked | 10 hCG | | | | | | | urine with |
| | | hCG | | | | | | | | 0.005% v/v |
| | | | | | | | | | | TWEEN 20 |
| | | | | | | | | | | and 4M |
| | | | | | | | | | | NaCl |
| 9* | 20 | Ab- | 10 Ab + | 1 | 75 | hCG | 10 | 1 | 45 | 6 × artificial |
| | | masked | 10 hCG | | | | | | | urine with |
| | | hCG | | | | | | | | 0.005% v/v |
| | | | | | | | | | | TWEEN 20 |
| | | | | | | | | | | and 4M |
| | | | | | | | | | | NaCl |

During the first phase of selection (rounds 1-5), a negative selection step designed to remove DNA sequences binding to the solid support beads was performed. During the second phase (round 6 to round 9), antibody-blocked hCG beads were used during counter selection, to remove aptamers binding other epitopes on the hCG molecule apart from the β₁ epitope.

The negative selection was performed using hCG coupled to magnetic beads via an antibody specific to the β₁-epitope. This strategy aimed to mask this epitope of interest in the negative selection step, where only unbound sequences are subsequently exposed to positive selection pressure, and thereby remove sequences binding to any other site of the target molecule from the ssDNA pool. Antibody masked negative selection was followed by positive selection against hCG-functionalised beads as in rounds one to five (FIG. 1). Counter selection against the related glycoprotein hormone, FSH, which shares an essentially identical α-subunit but contains a structurally- and functionally-unique β-subunit, was performed in the middle of each phase (third and seventh rounds) of selection to ensure identification of DNA aptamers specific to the β-subunit of hCG. The methods employed are more fully described below.

To ensure annealing of the complementary oligonucleotides and proper folding of the variable region of each library member, equimolar amounts of library and blocking oligonucleotides (Table 1) were mixed in an artificial urine matrix. This solution was heated to 95° C. for five minutes then slowly cooled to room temperature at a rate of 0.5° C. per minute in a 3Prime thermocycler (Techne), before incubation with functionalised magnetic beads.

Magnetic beads were coupled to the relevant protein (Table 2) according to the manufacturer's instructions. After protein incubation, any residual excess protein was removed by collecting the beads using a magnetic separator and aspirating the supernatant. The beads were washed four times with 1 ml 0.15 M HEPES, pH 7.0. Beads were blocked with 0.2 M Tris, pH 8.5 for one hour at 4° C., washed four times with HEPES then used immediately for library pool exposure. For rounds 6 to 9, hCG was incubated with the antibody-coated beads and allowed to bind for two hours at room temperature, with gentle rotation.

After four HEPES washes, the beads were resuspended in 1 ml of artificial urine matrix containing the folded, blocked library pool and incubated at room temperature with gentle rotation (see Table 2 for DNA quantities and incubation times). The dissolved library was then removed from the beads by magnetic separation and the DNA supernatant of the negative selection rounds was immediately transferred to the prepared positive selection beads.

During positive selection, the ssDNA pool remaining in the supernatant was allowed to bind to the target beads at room temperature for between 45-60 minutes, depending on the round (Table 2). During the counter selection, occurring at rounds 3 and 7, the resultant supernatant, after FSH binding, was collected and precipitated. For all other positive selection rounds, the supernatant after target binding was removed and any non-specifically or weakly bound sequences remaining on the beads were washed off with regiments that were increasingly stringent as SELEX proceeded (Table 2).

Tightly-bound sequences remaining on the target beads were finally eluted with two additions of 200 μl elution buffer (40 mM Tris, 10 mM EDTA, 3.5 M Urea, 0.002% TWEEN® 20, pH 8.80, while heating to 80° C. for ten minutes each. Eluted ssDNA was recovered by ethanol precipitation and resuspended in 30 μl H₂O.

Portions of the recovered DNA pool were briefly amplified by PCR for two cycles to generate dsDNA, for improved stability. The ssDNA was amplified using KAPA Taq (Sigma), with 0.25 μM of each primer, using the phosphorylated reverse primer. To generate sufficient copies of the recovered sequences for use in the following SELEX round, the DNA was further amplified using the same conditions. Amplification was stopped at an optimal number of amplification cycles (i.e. the number of PCR cycles generating maximal product but before higher molecular weight amplification artefacts were visible on a GelRed (Biotium) stained 10% PAGE gel). The ssDNA pool for the next round of selection was regenerated from the amplified product using 50 U lambda exonuclease (NEB) at 37° C. for one hour.

Monitoring SELEX Using qPCR

The quantity of DNA (as well as some preliminary analysis of sequence diversity) eluted directly from the magnetic beads, for both positive and negative selection steps at each round, was estimated using qPCR. Quantification of retained DNA at each selection round is often used as a method to monitor SELEX progress (Mencin et al., 2014). However, these reports do not generally consider any differences in target availability at individual rounds, nor do they (generally) report on quantities other than those obtained from positive selection.

In the present invention, ssDNA eluted from both positive and negative selection beads, at each SELEX round, was directly quantified by qPCR using Quantinova SYBR Green Master Mix (Qiagen) and 3.5 μM of each primer. Real-time amplification was performed in a CFX Connect™ Real-Time PCR Detection System (BioRad). The thermal cycling conditions used were: initial denaturation at 95° C. for 2 minutes, then 40 cycles each of denaturation at 95° C. for 5 seconds, annealing at 64° C. for 20 seconds and extension at 72° C. for 10 seconds.

The DNA pool diversity of each selection round was also assessed by examining the shape of the amplification plots and melt curves obtained from the qPCR quantification of the exonuclease digested samples from each round. Melt curve analysis was performed by cooling the amplicons to 55° C. after the final extension step, then gradually heating to 95° C. and monitoring fluorescent intensity. C_{q} values obtained from the CFX Manager Software were used to construct standard curves based on diluted library samples of known concentration, included on every individual plate.

The binding affinity of the ssDNA pool at various SELEX rounds was estimated by quantifying the amount of DNA bound to hCG-functionalised beads directly. Briefly, 1 pmol of exonuclease digested ssDNA, with equimolar amounts of the complementary blocking oligonucleotides, was heat-denatured and encouraged to form three-dimensional structures as in the SELEX experiment. The complemented and folded DNA was introduced to 1 mg of magnetic beads functionalised with 10 μg of hCG. After binding for one hour at room temperature with gentle rotation, the beads were collected and the supernatant containing any unbound DNA sequences was removed. After resuspending the beads in 1 ml fresh artificial urine matrix, 1 μl of the resuspended beads were used directly as the template in qPCR for DNA quantification. This was compared to the quantification of the original input DNA and the unbound fraction in the supernatant.

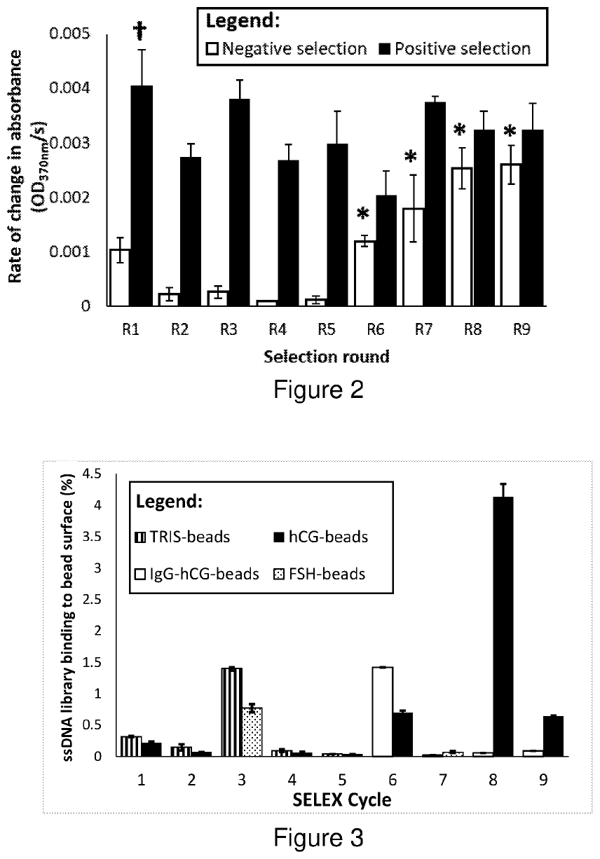

To account for any variance in target loading during selection, especially considering the change in selection pressure used in this SELEX strategy, aliquots of the protein-bound beads used in SELEX were analysed using an α-subunit antibody-based ELISA method to detect both hCG and FSH coupled to the magnetic beads (FIG. 2). Except for the first selection round—which used five times more beads than any of the other rounds (Table 2)—no difference in the amount of target protein present on the magnetic beads for the positive selection samples (Tukey post-hoc test, FIG. 2). The consistency of TMB signal produced between positive selection rounds indicates that while some batch-to-batch variation does occur between positive selection bead sample for each round, this difference is not statistically significant, suggesting that equivalent protein amounts were present throughout selection, even between the first (Rounds 1-5) and second phases (Rounds 6-9).

With the convergence of the DNA pool through selection, a concomitant increase in binding affinity for the target, hCG, is expected. The enrichment of target-specific DNA sequences was monitored through the selection process by incubating a small fraction of each ssDNA pool with hCG-functionalised beads. Bound DNA was quantified, on the beads, by qPCR and compared to the amount of input DNA used for each round (FIG. 3). Initially, only a small proportion of the large heterogeneous oligonucleotide library bound to the beads modified with the hCG target (0.219±0.019% of the input DNA), which decreased after the quantity of protein-loaded beads was reduced for Rounds 2-5 (˜0.07% of the initial pool). This value remained low for the early SELEX rounds before β₁-specific selection was introduced, before increasing to a maximum of 4.13±0.21%) by the end of round 8.

Generally, in the first phase of selection (Rounds 1-5), more ssDNA remained bound to the magnetic beads used in the negative selection step than to the positive selection beads (FIG. 3). However, once the selection pressure was changed from the sixth round, more DNA was eluted from the positive selection beads than those used in the negative selection step. As selection progresses, more of the DNA pool is expected to be recovered from the positive selection steps due to improving affinity of the DNA pool towards the target and between rounds seven and nine, more DNA is retained on the positive selection beads than the antibody masked hCG-functionalised beads used for negative selection, even with the increasing stringency applied through selection (Table 2). These findings show the changing behaviour of the DNA pool through selection, indicating an increased affinity towards the target as SELEX progressed.

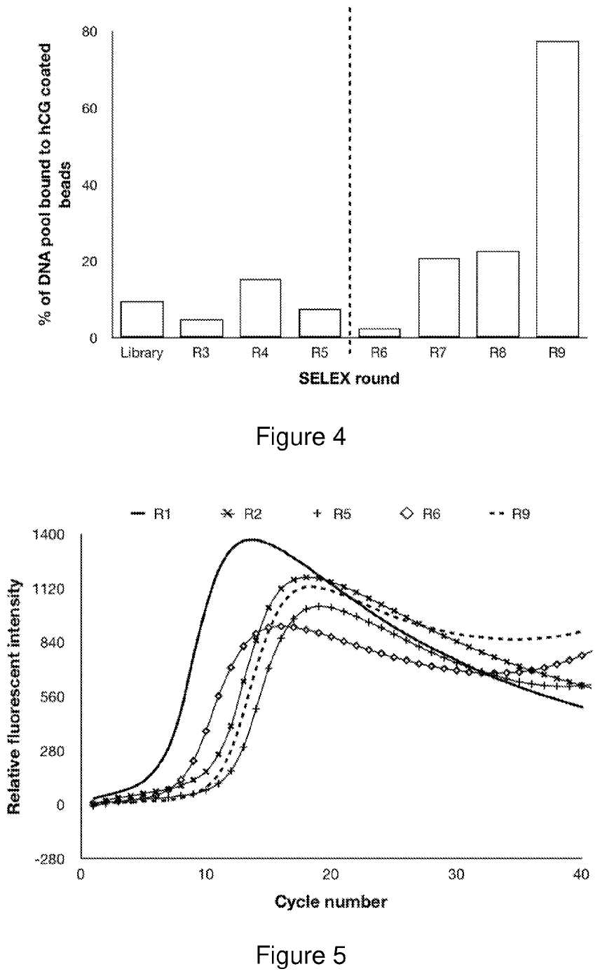

To validate the findings obtained during SELEX; and to circumvent assays based on the different quantities of proteins on the different batches of beads (FIG. 2, Table 2), the different bead quantities used (Table 2) and the different washing stringencies (Table 2), a post-SELEX assay was conducted (FIG. 4). This was conducted similarly to the SELEX process: a single batch of hCG-modified beads was created (10 μg hCG/mg beads). To 1 mg of these beads, 1 pmol of ssDNA obtained from samples reserved from each round of SELEX was exposed. ssDNA bound to the beads was then quantified with qPCR. A dramatic drop in pool affinity was seen after the sixth round (only 2.54% of the DNA pool bound to the target-coated beads), once the β₁-epitope masked hCG complex was included in the negative selection steps. This is likely due to the rapid change in selection pressure introduced at this stage of the SELEX strategy. While an increasing proportion of the DNA pool bound to the target beads after counter selection at round 7 (20.88%) and positive selection at round 8 (22.85%), by round 9, the majority of the input DNA bound to the target (77.39%), a level comparable to previous reports (Park et al., 2016).

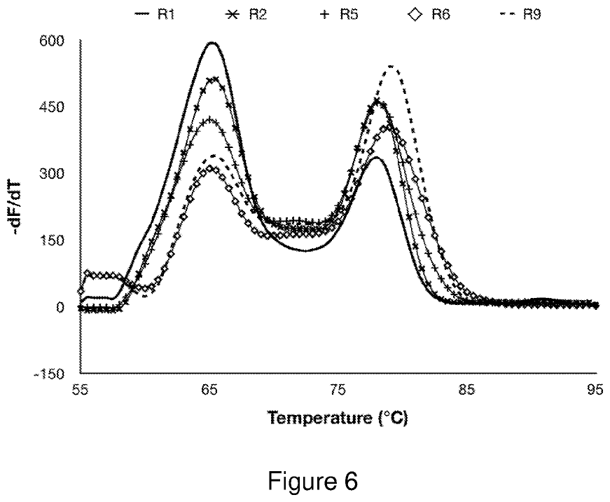

In addition to quantification of the recovered DNA pools through selection, qPCR analysis of the exonuclease digested samples from each SELEX round was used to monitor changes in pool diversity by simply examining the amplification plots (FIG. 5) and melting profiles (FIG. 6). Recently this method has been used extensively to show decreasing diversity through selection by monitoring the change in shape of these plots.

The distinctive dip in fluorescent intensity with increasing qPCR cycle numbers, after peak fluorescence is reached, characterises the highly complex and diverse DNA pools of the early selection rounds. However, as selection progresses, this decrease becomes less pronounced, indicating convergence of the sequences present in the DNA pool at later rounds of SELEX (FIG. 5). In early selection rounds, where sequence diversity is highest, mismatched and unstable hetero-duplexed DNA sequences readily form and result in a decrease in fluorescence as they dissociate during the conventional plateau phase of homogeneous template qPCR and thereby release the intercalated fluorescent dye at lower temperatures in the amplification cycle where the fluorescent signal is acquired. However, as selection continues and pool diversity decreases, this DNA mismatch is reduced and results in the disappearance of the drop in fluorescence.

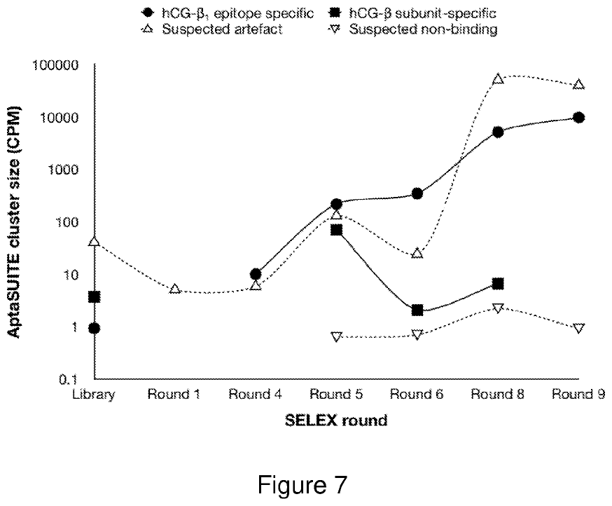

Additionally, the shape and position of the peaks of the melting profiles change as SELEX proceeds (FIG. 7) and indicate convergence of the DNA pool at later selection rounds. Hetero-duplexed amplicons, characterised by a melt peak at a lower temperature, centred around 67° C., predominate at the earlier selection rounds. However, by the end of selection at round 9, homo-duplexed DNA sequences showing a defined peak at the higher melt temperature of 81° C., predominate the pool (FIG. 6). Taken together, these results indicate sufficient convergence of the DNA pool and progressive enrichment in the DNA population of strong hCG binding sequences by the ninth round of SELEX, hence selection was concluded. Moreover, qPCR techniques provide a quick, straightforward and affordable method to not only quantify DNA through SELEX but also estimate pool diversity.

### EXAMPLE 3

### Next Generation Sequencing (NGS) and Analysis

NGS libraries for MiSeq sequencing (Illumina) were generated from recovered pools from various SELEX rounds (1, 4, 5, 6, 8 and 9), as well as the initial, unselected library. Illumina-specific primers were ligated onto each sample pool by reacting a 1 μl aliquot of the pool with 0.25 μM of each Illumina-specific primer. The forward (F) and reverse (R) primer sequences were as follows:

F:
(SEQ ID NO: 14)
5′-TCGTCGGCAGCGTCAGATGTGTATAAGAGACAGTCGCACATTCCGCT
TCTACC-3′
R:
(SEQ ID NO: 15)
5′-GTCTCGTGGGCTCGGAGATGTGTATAAGAGACAGTTCGCACACACGG
ACTTACG-3′

where the underlined nucleotides correspond to the constant primer binding regions of the SELEX pool.

After PCR amplification and purification of the desired amplicon via gel excision from a 2.5% w/v agarose gel, the purified 147 bp product was submitted to the NRF-SAIAB Aquatic Genomics Research Platform (Grahamstown, South Africa) for Illumina library generation and paired-end 75 bp MiSeq sequencing. All samples were multiplexed with unique barcodes for individual rounds to allow for them to be combined and sequenced together on a single MiSeq flow cell.

Illumina paired-end FASTQ files were demultiplexed, based on the barcodes for each selection round analysed. Initially, each SELEX round was analysed separately. These demultiplexed reads, for each analysed round, were processed using AptaSUITE (Hoinka et al., 2018), using default parameters. Sequences were clustered using the AptaCLUSTER package (Hoinka et al., 2014) contained within the AptaSUITE collection and subsequently output as sequences ranked in order of frequency of occurrence within each pool within a text file, for further analysis.

The frequency-ranked lists of unique sequences for each round were assigned a name to each sequence, depending on which round they initially appeared in, and their position in the ranked list (for example R4_1 was the most abundant sequence identified in the fourth round of SELEX). These lists could then be interrogated to identify persistent sequences and track their abundance through selection.

Candidate aptamers were chosen based on their abundance and enrichment through selection and their response to changing selection parameters, relative to control library pools.

Seven ssDNA pools from key selection rounds (R1, R4, R5, R6, R8 and R9, in addition to a sample of the starting library), were analysed by next-generation sequencing (NGS) to investigate their sequence composition and evolution through the targeted selection strategy. The seven individual samples were uniquely barcoded to allow parallel, multiplexed sequencing on a single Illumina MiSeq flow cell.

Over 1 million paired-end reads were obtained for each sequenced sample, with over 10 million raw reads acquired in total. On average, 94.48%±0.36 of reads for each sequenced round passed AptaSUITE quality controls, after demultiplexing the barcodes, merging the paired-end reads and trimming off primer sequences (Table 3).

Table 3: Comparison of numbers of NGS sequence reads obtained and analysed using the AptaSUITE bioinformatic platform. The total number of raw reads initially obtained is shown for each sequenced round, as well as the numbers of filtered sequences and unique sequences obtained through analysis. Percentages of the filtered sequences for unique reads are shown. The enrichment of the pool (the inverse of sequence complexity) was calculated as a percentage of the number of unique reads, relative to the total filtered reads.

| SELEX | Initial | Merged, trimmed | | |
|---|---|---|---|---|
| round | reads | and QC filtered | Uniques (%) | % enrichment |
| Library | 1136316 | 1076687 | 98.79 | 1.21 |
| Round 1 | 1867344 | 1770915 | 99.14 | 0.86 |
| Round 4 | 1263374 | 1196968 | 94.81 | 5.19 |
| Round 5 | 1682308 | 1590013 | 83.71 | 16.29 |
| Round 6 | 1532961 | 1443487 | 69.37 | 30.63 |
| Round 8 | 1439972 | 1360272 | 46.10 | 53.90 |
| Round 9 | 1172576 | 1100523 | 51.76 | 48.24 |

The number of unique reads present in each sequenced sample steadily decreased through selection: from between 98.8% (library) and 99.1% (after one round of selection) to an apparent plateau of approximately 50% unique reads by the end of SELEX (Round 8: 46.10%, and Round 9: 51.76%). Concomitantly, the copy number of individual AptaSUITE seed sequences (i.e. the abundance of specific sequences appearing in the SELEX pool samples) increased through selection. Together, these demonstrate a decreasing heterogeneity of the pool expected for the enrichment of particular sequences through SELEX (Table 3) and predicted by the initial qPCR studies (FIGS. 3-6).

With the unique, two-phase selection strategy used here, the predicted binding characteristics of sequences selected at various rounds of SELEX, could be identified. Four broad groups of binders could be characterised by monitoring the changes in their cluster size as selection progressed (FIG. 7). The first group are sequences binding to the β1 epitope of hCG (“hCGβ1 epitope-specific”) which should only become significantly enriched between rounds 6-9. Sequences binding to the β subunit of hCG, but definitely not at the β1 (“hCGβ subunit-specific”) would enrich between rounds 1-5 but begin to decline in frequency following the change in target in round 6. Correspondingly, sequences showing sustained increasing enrichment throughout selection, irrespective of the change in target, were assigned to be sequences that arise due to preferential amplification during PCR between SELEX rounds, rather than binding to the target (“suspected artefact” sequences). Additionally, sequences showing no enrichment from the start to end of SELEX are generally assumed to be poor target-binding sequences (“suspected non-binding”).

Abundance of selected example sequences was monitored through SELEX by examining the AptaSUITE cluster size at each sampled round, with expected binding behaviour under changing selection pressure used to identify different categories of potential hCG binders.

NGS analysis of the initial, unselected library revealed the presence of some predominant sequences (approximately 1.2% of the sample), suggesting a biased solid-phase synthesis process resulting in reduced library diversity and complexity. Although SELEX is often assumed to begin with a random population of unique DNA sequences, NGS studies of unselected libraries have shown this not to be the case. The overrepresented sequences present in the library used here accumulated through selection and dominated the sequenced pools (partially represented by the “suspected artefact” sequence, FIG. 7), indicating that the target and selection conditions employed in this SELEX strategy favoured their maintenance and amplification. These parasite sequences showed comparable behaviour, through SELEX, to what would be expected from a sequence capable of binding the 1 epitope of hCG, increasing generally throughout SELEX and reaching maximal abundance by the end of selection, despite the temporary decrease in their enrichment between Rounds 5 and 6 (FIG. 7). Without comparison to the starting library and monitoring the change in sequence abundance under changing selection pressure, these sequences would have been indistinguishable from highly enriched and abundant candidate aptamers. Therefore, altering the selection pressure midway through SELEX proved to be a useful and informative method for identifying amplification artefacts which would otherwise have steadily increased in prevalence through selection.

### EXAMPLE 4

### Screening of Aptamer Candidates

From the most abundant and most highly enriched sequences in each NGS predicted binding category, five unique sequences were chosen and empirically tested for target binding. The results of the most promising candidates from each of the four different predicted binding categories (excluding a non-binding sequence) are included here. These sequences were screened for their individual binding abilities to hCG, using a variety of molecular biology techniques. ELONA-style assays, requiring detection of target-bound aptamer, used 5′-biotinylated aptamer sequences without primer binding sites (i.e. truncated to include only the variable region). For qPCR screening, where no DNA modification was necessary and primer binding sites were used for amplification, full-length aptamer sequences were tested. The available aptamer format (full-length, unmodified Lib_1 and 5′-biotinylated, truncated R4_1, R4_64, R5_4 and R6_5) was used for EMSA screening.

The binding affinity of these novel sequences was investigated using a variety of assays and conditions. These assays involved differing states of the hCG target and the screened aptamers, to assess whether screened sequences were capable of consistently binding selectively to the chosen targets, as more fully described below.

Aptamer candidates were diluted to 250 nM in artificial urine and secondary structure formation was encouraged by heating to 95° C. for ten minutes, followed by gradual cooling to room temperature for at least ten minutes. hCG, dissolved in 0.15 M HEPES pH 7.0 (27 pmol), was then added to 2.5 pmol (10 μl) of the folded aptamer and incubated at room temperature for one hour. The entire sample was then electrophoresed on a 10% v/v PAGE gel at 80 V for 80 minutes. The separated DNA bands were visualised after 20 minutes of GelRed staining using a BioRad Gel Doc™ EZ Imager. The pixel intensity of each band before and after hCG addition was quantified using ImageJ software. An hCG only sample (27 pmol) without addition of any DNA was included as a negative control, while 2.5 pmol folded aptamer, in the absence of hCG, showed the electrophoretic migration profile of individual aptamers.

Protein-modified magnetic beads were prepared by functionalising 1 mg of epoxy beads with either 10 μg of hCG or negative control proteins BSA or HSA (as in Example 2). Once functionalised, aliquots of the beads corresponding to 72 μg beads were added to individual wells of a 96-well plate which were pre-blocked with 3% w/v milk powder (in HMCKN-T buffer composed of 20 mM HEPES, 2 mM MgCl₂, 2 mM CaCl₂, 2 mM KCl, 150 mM NaCl, pH 7.4, supplemented with 0.002% v/v TWEEN 20.) Bead samples were prepared in triplicate.

After two HMCKN-T washes, 25 pmol of folded DNA aptamer solution in artificial urine was added (prepared as for the electrophoretic mobility shift assays, Section 2.2.4) to each well. The aptamers were incubated with the functionalised beads for one hour at room temperature then each well was washed three times with 200 μl HMCKN-T.

Subsequently, the biotin modification on retained aptamers was used to immobilise SA-HRP (KPL), diluted 1:5000 in HMCKN-T and incubated at room temperature for half an hour. To quantify immobilised SA-HRP, the colorimetric HRP-catalysed TMB oxidation reaction was initiated by adding 50 μl of 1-step Ultra TMB-ELISA substrate (ThermoScientific) to each well. TMB oxidation proceeded at room temperature, in the dark, for half an hour and thereafter absorbance was measured at 370 nm and 650 nm, according to the manufacturer's instructions.

For each microtitre plate used in ELONA, DNA-free control tests were included to account for non-specific interaction with the protein-modified beads and to accommodate signal variation between plates arising from minor variations in HRP loading and TMB incubation times.

To assess the concentration dependence of the assay response on the amount of protein target present, and estimate the kinetic behaviour, the same procedure as above was conducted, using a range of bead quantities per well (200 μg, 50 μg, 12.5 μg, 3 μg and 0 μg).

In order to determine the epitope specificity of some of the aptamer candidates, antibodies with known hCG binding sites (either (1-epitope or α-subunit) were used to competitively elute aptamer sequences sharing the same, or overlapping, binding sites. The difference in assayed biotin content between antibody-eluted and uneluted aptamer: bead samples was then assayed.

For these experiments, 100 μg of protein-functionalised beads was added to each well and incubated with 2.5 mM folded, biotinylated, aptamer for one hour at room temperature. 100 μl of a 1:1000 dilution of the competing primary antibody, in HMCKN-T, was then added per well and allowed to bind for an hour. Following three HMCKN-T washes, the amount of biotin present was measured as described above. This was performed twice, with quadruplicate wells for each candidate aptamer and competition condition used, in each experiment. The results were then combined to give at least 8 replicates for each condition, increasing the statistical power.

An alternative competitive binding assay was used to determine any overlapping binding specificity between the candidate aptamers. For this experiment, full-length, unlabelled aptamer sequences were used to compete with the truncated, biotinylated versions of the sequences (Table 1). 250 nM biotinylated, truncated, folded aptamer sequence was incubated with 80 μg protein-functionalised beads and allowed to bind at room temperature for one hour. A mixture of sequences comprising 5 μM unlabelled full-length aptamer, as well as 5 μM of each complementary blocking oligonucleotide for the constant regions, in artificial urine, were combined and folded. After three HMCKN-T washes, 100 μl of the unlabelled, competing sequence was added to the beads previously exposed to biotinylated aptamer. Again, biotin quantity was detected with SA-HRP, as described above, and compared to bead samples containing biotinylated aptamers not exposed to full-length, unlabelled sequences.

To test aptamer candidates' ability to capture unimmobilised hCG, streptavidin-coated beads were prepared using 10 μg of streptavidin (Sigma) per mg magnetic beads and ELONA plates were prepared as described above using 60 μg of functionalised beads per well.

Folded, biotinylated aptamers (prepared as above for the EMSA and magnetic bead ELONA) were immobilised onto the streptavidin-functionalised beads for 30 minutes at room temperature. Beads were washed three times with HMCKN-T and incubated with 100 μl of a 1 μg/ml hCG dilution in HMCKN-T for an hour at room temperature. hCG captured on the aptamer-modified beads was detected using a 1:5000 dilution of the α-subunit specific primary antibody in HMCKN-T. After 30 minutes at room temperature, and three HMCKN-T washes, a 1:5000 dilution of the HRP-conjugated secondary antibody was added and allowed to bind for half an hour at room temperature. Following three HMCKN-T washes, colorimetric TMB oxidation was developed as described above. DNA-free and unbiotinylated DNA control tests were included as negative controls.

A low-cost paper surface was used to immobilise hCG and investigate candidate aptamers' ability to detect the target in this system. Whatman chromatography paper (grade 1, GE Healthcare Life Sciences) was used, modified with printed wells. The printed wells were 5 mm in diameter and surrounded by a 2.12 mm-wide hydrophobic wax barrier, printed onto the paper surface with a Xerox Colorqube 8870 printer. Before use, the wax was melted into the paper by heating to 100° C. on a hotplate for a few minutes, creating a hydrophobic reaction well to contain the reaction components within a small area during testing.

hCG was immobilised onto the surface of each well using EDC/NHS coupling. 0.1 M NHS and 0.4 M EDC stocks were prepared fresh in 0.15 M HEPES, pH 5.8. A 5 μl spot of freshly-mixed 1:1 EDC/NHS solution was applied to the centre of each wax-printed well and allowed to react for 15 minutes at room temperature. This was repeated with another 5 μl EDC/NHS aliquot. Each well was individually washed, three times, with 5 μl HEPES buffer, wicking excess fluid onto fresh paper toweling placed underneath the paper. 5 μl of 20 μg/ml hCG solution, diluting the stock solution in HEPES, was added to the paper wells and incubated at room temperature in a humidified chamber for one hour. Each well was subsequently washed, three times, with HEPES buffer. 2 μM dilutions of heat-treated, biotinylated aptamer sequences, in artificial urine, were prepared, 2 μl of this DNA solution was added per well and incubated at room temperature for two hours. Each well was subsequently individually washed, three times, with artificial urine.

The entire sheet of wax-printed paper was then submerged in 3% w/v milk powder in PBS-T and incubated under rocking for at least an hour at room temperature, to block the paper surface from non-specific binding. The entire sheet was washed by submerging it into HEPES buffer three times.

Biotinylated aptamers were detected using a polyclonal goat anti-biotin antibody (Abcam). The HRP-conjugated antibody was diluted 1:10000 times in the milk powder block and 5 μl was added to the centre of each paper well. After half an hour of incubation, the entire sheet was submerged in PBS-T for three washes and briefly air-dried on paper towelling. 5 μl of TMB substrate (SeraCare) was added per well and an image of the colour development was immediately captured using a desktop scanner (Cannon CanoScan LiDE 110). Quadruplicate wells used for each condition.

The pixel intensity of the interior of each well was calculated using ImageJ. Briefly, an RGB image file was split into the three colour channels and inverted. Each well was individually selected and added to the OI manager in ImageJ. The intensity of each spot, in each colour channel, was then measured and these values imported into a spreadsheet for analysis. The average value of the blank well (“No DNA” control) in each colour was subtracted from every other well and squared. The change in intensity (ΔI) for each well was calculated from the Euclidean distance, the square root of the sum of the squares of RGB channels, as previously described for paper-based ELISA detection systems.

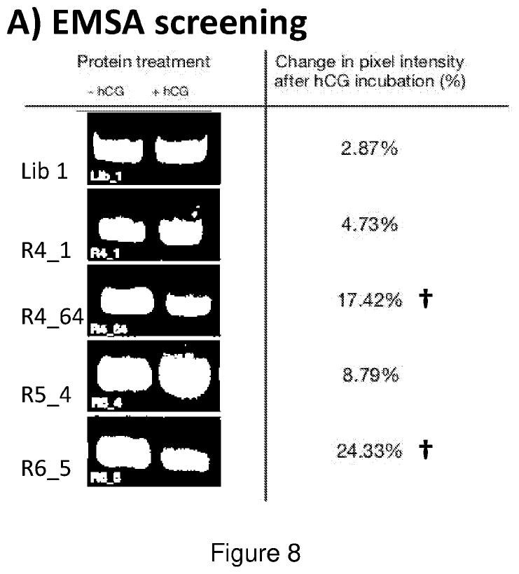

EMSA (FIG. 8), allowed both the hCG target and the aptamer candidates to bind freely to one-another in solution. This was selected as a screening approach, as aptamer immobilisation and/or the conjugation of functional agents to the sequence has previously been shown to influence binding performance. In EMSAs, target binding was assessed by incubating the target with the aptamer to form aptamer-target complexes, subsequently separating the unbound aptamers from the aptamer-target complexes by electrophoresis on a 10% w/v PAGE gel. The formation of aptamer-target complexes is anticipated to decrease the band associated with unbound aptamer fraction. Aptamer-target binding was thus evaluated by comparing the band intensity of the band associated with unbound DNA aptamer band in the absence of the target (“aptamer alone” samples in FIG. 8), to the same band after incubation with a ten-fold molar excess of hCG (“aptamer and hCG” samples in FIG. 8). For this study, a decrease in band intensity of at least 10% in the presence of hCG was taken as evidence of significant binding.

The sequences predicted by the NGS data to be potential amplification artefact sequences, Lib_1 and R4_1 (FIG. 7) showed very slight decreases in DNA band intensity after hCG addition during EMSA evaluation (FIG. 8), indicating that little of the DNA had formed hCG-aptamer complexes. These results confirm their poor target-binding affinity and suggest their maintenance and amplification through selection resulted from initial library bias after which they evolved to dominate the DNA pool under the selection conditions used. In contrast, sequences R4_64 and R6_5 showed visible decreases in the intensity of the DNA band associated with unbound aptamer; these quantified as decreases of between 17 and 24%, respectively. Sequence R5_4 showed potential binding ability, but not strong binding ability, when assessed by this method. These results provided initial confirmation of target binding ability for some of the novel aptamer sequences, which were verified using other conventional aptamer-binding assay techniques.

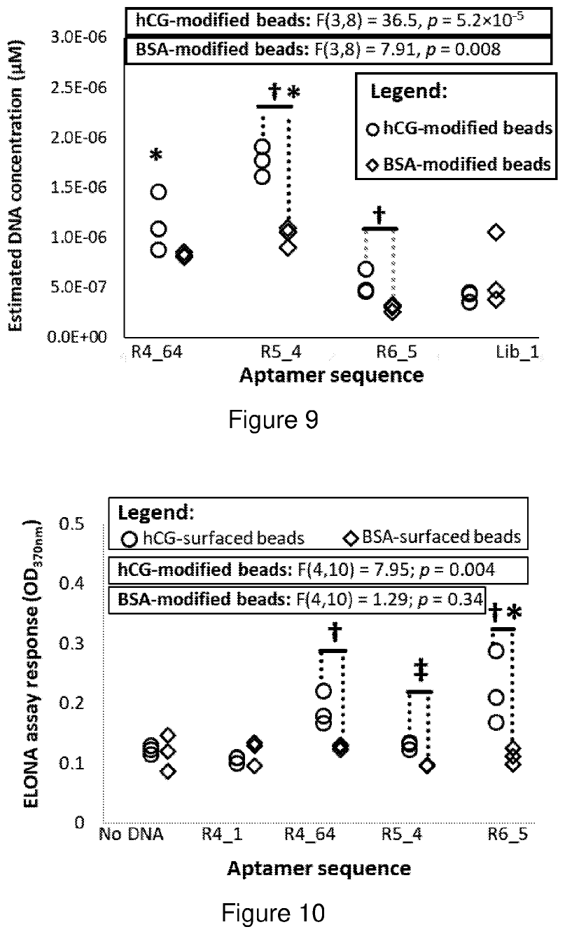

In a study designed to mimic SELEX conditions, the selected sequences were screened for their ability to bind to hCG-surface magnetic beads, subsequently detecting the bound sequences by qPCR (FIG. 9). Magnetic beads modified with Human Serum Albumin protein (HSA) were used as control surfaces. Similar to EMSA, unmodified, full-length DNA sequences were tested for binding to hCG-conjugated beads (or HSA-coated beads as a non-specific control) by qPCR. As a marker of kidney function, HSA was selected as a control protein due to its expected presence in large amounts within urine samples. To replicate the experimental conditions used during selection, and limit aptamer fold and function to only the variable region, the aptamer candidates were heat denatured and re-annealed in the presence of oligonucleotide blockers complementary to the primer binding sites.

Binding of the tested sequences to hCG-coated beads was significantly influenced by the sequence of the aptamer used, with the use R4_64 and R5_4 resulting in significantly more DNA bound to the target beads than the control Lib_1 sequence (FIG. 9). All tested aptamers (Besides Lib_1 (t(4)=−1.04, p=0.36)) showed a higher affinity for hCG-modified beads, compared to beads modified with HSA. Preferential binding to hCG was significant for aptamers R5_4 (t(4)=7.23, p=0.0019) and R6_5 (t(4)=3.29, p=0.030) but not R4_64 (t(4)=1.85, p=0.14).

A variation of the qPCR assay (FIG. 9) was employed using biotinylated aptamer sequences, creating a colourimetric ELONA (FIG. 10). During the colourimetric ELONA study, 5′ biotinylated full-length aptamer sequences were exposed to protein-modified beads—either hCG target or Bovine Serum Albumin (the bovine equivalent of HSA). After rinsing unbound aptamers from the beads, the colorimetric signal, proportional to the amount of biotinylated aptamer present, was generated by the addition of HRP-conjugated streptavidin and the subsequent HRP-catalysed oxidation of TMB.

Similar to the EMSA and qPCR results, ELONA-based screening of the aptamer candidates (FIG. 10) identified sequences R4_64 and R6_5 as capable of binding to hCG, as well as lower levels of R5_4 binding. The amplification artefact sequence R4_1 was selected as a negative control and exhibited low levels of binding to either protein, similar in extents to the “No DNA” control samples. Similarly, none of the sequences exhibited significantly different binding to BSA-modified beads, compared to the “No DNA” sample, indicating a lack of binding to this protein (ANOVA annotation, FIG. 10). Relative to their BSA control bead samples, all three tested aptamers (R4_64; R 5_4 and R6_5) demonstrated significant binding to hCG-modified beads (†): R4_64 (t(4)=3.82, p=0.019), R5_4 (t(4)=8.53, p=0.001) and R6_5 (t(4)=3.07, p=0.037). Furthermore, sequence R6_5 exhibited a significantly higher colorimetric signal compared to the No DNA control sample (*); higher (but not significantly higher) responses were evident for R4_64 (FIG. 10).

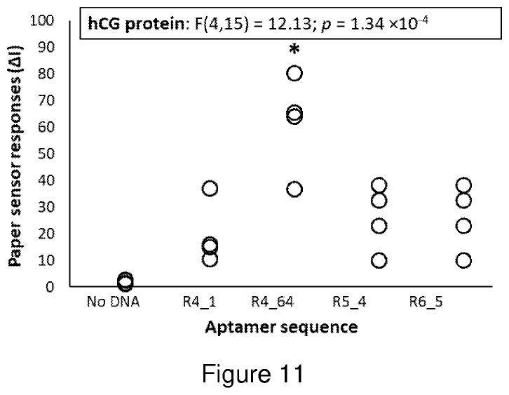

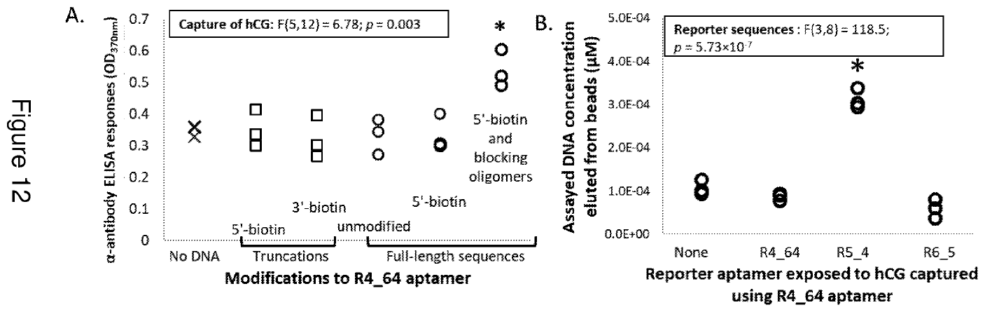

An alternative binding approach was investigated using a low-cost paper surface on which the hCG protein target was immobilized using EDC/NHS coupling to the cellulose. As with the bead-based assay, the amount of biotinylated aptamer present was considered to be proportional to the colorimetric signal generated by TMB, resulting in a blue colour within the wells at the end of the assay (FIG. 11, top panel). Using this approach, all sequences tested showed increased signal compared to the “No DNA” control, however, only sequence R4_64 generated significantly more colorimetric signal than the R4_1 sequence (* in FIG. 11). These results are in agreement with the previous binding assays and indicate that R4_64 is able to recognise and bind to hCG. They also emphasise the suitability of particular sequences to particular techniques and the need for investigating multiple methodologies when screening novel aptamers.

Viewed overall (FIGS. 8-11), the comprehensive screening techniques employed indicate that the aptamer sequences identified from NGS analysis: R4_64, R5_4 and R6_5; consistently bind to the hCG target. Overall, the consistency of binding the different sequences could be evaluated as significant/specific binding in the following order: sequence R4_64 (which exhibited significant/specific binding for all assays used here), followed by both sequences R6_5 and R5_4 (which each demonstrated assay-dependent binding). This highlights the NGS-based rational approach for selection of sequences based on enrichment during SELEX.

Correspondingly, the sequences identified by NGS analysis as unlikely to bind the target to a great degree showed expected results. The Lib_1 sequence demonstrated minimal binding when evaluated using EMSA (FIG. 8) and qPCR. This sequence showed the least amount of DNA bound to hCG-functionalised beads (FIG. 9). Similarly, the amplification artefact sequence R4_1, which showed very similar behaviour to Lib_1 through selection during NGS evaluation (FIG. 7), displayed little evident binding to the target by the assay techniques used here (FIGS. 8, 10 and 11). Viewed together, these results suggest that this group of sequences were maintained in the SELEX pool as a consequence of their ready amplification during PCR, rather than their target binding specificity. By thoroughly examining the evolving selection pool through SELEX by NGS, from the very beginning by including the unselected library, these anomalous high-frequency but low-affinity sequences can be identified. This analysis can then exclude any parasite sequences from potential aptamer validation experiments, which can be costly and labour-intensive.

### EXAMPLE 5

### Identification of hCG Aptamers Specific to Distinct Sites of the Target Molecule

To determine whether the targeted selection strategy employed here allowed for the successful identification of aptamers specific to different regions of the protein target, competitive molecular biology assays were used. Exploiting the known binding sites of the available commercial antibodies allowed competition assays to be used where aptamer sequences sharing these sites would be eliminated and therefore reduce the colorimetric signals reported by the SA-HRP detection system as less biotinylated aptamer would be present. On the basis of consistency of binding (FIGS. 8-11), R4_64 was selected for further study, evaluating its ability to capture the target, via a bead-based ELISA assay. Variations of the same sequence (both full-length sequence and truncations of the variable region of the aptamer and 5′ and 3′ biotin modifications) were immobilised through their biotin modifications onto streptavidin-coated beads, which were exposed to hCG. Bound hCG was subsequently detected using an antibody specific to the α-subunit of hCG.

Briefly, magnetic beads, coated either with hCG or HSA, were functionalised and 50 μg beads were added per well of a 96-well microtitre plate and washed twice with HMCKN-T. A volume of 100 μl of a 250 nM dilution of folded, unlabelled, full-length aptamer sequences were added per well containing 25 μl of protein-functionalised beads. Binding was allowed to occur at room temperature for an hour, after which each well was washed three times with HMCKN-T (0.05% v/v TWEEN 20), once with HMCKN and resuspended in 50 μl HMCKN. 1 μl of this suspension was used as the DNA template in a SYBR green real-time PCR amplification, as described above.

To test whether aptamer sequences could be combined in a sandwich format, streptavidin coated beads were prepared and 25 μl of the 2 mg/ml bead suspension was added per well of a 96-well plate. 250 nM biotinylated full-length R4_64 DNA was folded in the presence of each complementary blocking oligonucleotide, in artificial urine, and 100 μl added per well and allowed to bind at room temperature for an hour. After three HMCKN-T washes, 100 μl of 1 g/ml hCG was added per well and incubated at room temperature for one hour. Each well was washed three times with HMCKN-T, once with HMCKN and resuspended in 50 μl HMCKN. 1 μl of this bead resuspension was used as the template in a qPCR assay to quantify the amount of unbiotinylated DNA bound to the captured hCG target.

Only when the full-length sequence that was 5′-biotinylated aptamer and folded in the presence of the complementary oligonucleotides was used, was a detectable, statistically significant colorimetric signal produced (* in FIG. 12A). This oligonucleotide was therefore able to capture and immobilise free hCG from the solution, but only when the aptamer was prepared in a configuration identical to the conditions experienced during SELEX. These results suggest that the correct folding of this aptamer depends on the primer binding sites being heteroduplexed and therefore unavailable for binding with the internal random region. The attachment of the biotin reporter at the end of this inert double-stranded region may also allow enough room to avoid steric hindrance when the DNA molecule is immobilised onto the magnetic bead. It has previously been shown that including a spacer region between the immobilised aptamer and the solid surface improves correct folding and minimises steric hindrance, with the optimal spacer configuration crucial for immobilised aptamers' target binding ability, which is supported by the data presented here.

Having demonstrated the ability to capture hCG, oligomer-blocked R4_64 was used as the capturing aptamer to investigate whether any of the other aptamer candidates could be combined with it to detect hCG, equivalent to an antibody sandwich assay. Beads containing hCG captured by R4_64 were exposed to full-length reporter aptamer sequences that were also folded in the presence of the blocking oligonucleotides. The DNA on the beads (assumed to be a combination of the R4_64 capture aptamer and the screened reporter aptamers) was eluted and quantified by qPCR (FIG. 12B). As expected, the use of folded R4_64 aptamer as a reporting agent exhibited no increase in the DNA yielded from the beads, indicating that the site exploited by the R4_64 during hCG capture is occupied. The similar DNA yield from the use of R6_5 also indicated a competition in binding sites between R4_64 and R6_5, indicating that both potentially bind at or near the 1 epitope of hCG. However, significantly more DNA was assayed when screening the R5_4 aptamer candidate as a capture agent: either when no sandwiching DNA was used (p=0.00023), or when R4_64 (p=0.00023) or R6_5 (p=0.00023) sequences were used to report on the presence of immobilised hCG.

Competition assay results (FIG. 12B) suggest that R4_64 and R6_5 aptamer candidates have the same, or highly overlapping binding sites on the hCG target molecule, possibly the β₁-epitope. R5_4 does not share this specificity, but rather appears to recognise a distant and distinct site on the β-subunit and can be used in combination with R4_64, and possibly also R6_5. To definitively ascertain where on the hCG molecule these sequences do bind, a biophysical technique is needed. X-ray crystallography, one of the most powerful tools for investigating the structure of aptamer target complexes, has previously been used to determine the crystal structure of an aptamer-protein complex to identify their exact interaction sites. More recently, the interaction interfaces of novel aptamers and their target protein have been simulated using molecular docking and molecular dynamics simulations to identify which amino acids of the protein are involved in aptamer binding. This would be useful in verifying the exact target binding sites of these hCG aptamers.

The competition between R4_64 and R6_5 was unexpected, since specificity of binding to the β₁ epitope formed a selective pressure from Round 6 of SELEX, onwards. Hence, it would be expected that DNA sequences originating from Rounds 6-9 would start to be enriched specifically against the β1 i.e. in these studies, sequence R6_5. Although R4_64 originated earlier in selection, before epitope specific selection was employed, the β₁-epitope was exposed and available for aptamer binding during all rounds of selection. This epitope comprises only approximately 4.5% of the surface area of the hCGB molecule, but any sequences binding to this region of the protein would be expected to increase in abundance throughout SELEX, even after the antibody-blocked hCG formed part of the negative selection step was introduced. Interestingly, when monitoring the DNA eluted from the SELEX beads at each selection round (FIG. 3), an increase in the DNA pool recovered from the positive selection beads is observed at round 6, where the selection strategy changed to include β₁-epitope specificity. This suggests that the previous five rounds of selection had already started to condition the pool towards this region of the protein target, with one such sequence possibly being R4_64.

### REFERENCES

Berger, P., Paus, E., Hemken, P.M., Stugeon, C., Stewart, W. W., Skinner, J. P., Harwick, L. C., Saldana, S. C., Ramsay, C. S., Rupprecht, K. R., Olsen, K. H., Bidart, J. M., and Stenman, U. H. 2013. Candidate epitopes for measurement of hCG and related molecules: the second ISOBM TD-7 workshop. Tumor Biology. 34:4033-4057.
Chutipongtanate, S. and Thongboonkerd, V. 2010. Systematic comparisons of artificial urine formulas for in vitro cellular study. Analytical Biochemistry. 402(1): 110-112.
Mencin, N., Šmuc, T., Vraničar, M., Mavri, J., Hren, M., Galeša, K., Krkoč, P., Ulrich, H., and Solar, B. 2014. Optimization of SELEX: comparison of different methods for monitoring the progress of in vitro selection of aptamers. Journal of Pharmaceutical and Biomedical Analysis. 91: 151-159.
Park, J -W., Lee, S. J., Ren, S., Lee, S., Kim, S., and Latrell, T. 2016. Acousto-microfluidics for screening of ssDNA aptamer. Scientific Reports. 6:27121.
Hoinka, J., Backofen, R., and Przytycka, T. M. 2018. AptaSUITE: A full-featured bioinformatics framework for the comprehensive analysis of aptamers from HT-SELEX experiments. Molecular Therapy-Nucleic Acids. 11: 515-517.
Hoinka, J., Berezhnoy, A., Sauna, Z. E., Gilboa, E., and Przytycka, T. M. 2014. AptaCluster—a method to cluster HT-SELEX aptamer pools and lessons from its application. Research in Computational Molecular Biology. 8394: 115-128.

1. An aptamer comprising or consisting of the nucleotide sequence of any one of SEQ ID NOs: 7-9, or a complementary sequence thereof,
wherein the aptamer selectively and/or specifically binds human chorionic gonadotropin (hCG).

2. The aptamer of claim 1, wherein the aptamer has the nucleotide sequence of any one of SEQ ID Nos: 11-13, or a complementary sequence thereof.

3. The aptamer of claim 1, wherein the aptamer binds the β subunit of hCG.

4. The aptamer of claim 3, wherein the aptamer binds the β₁ epitope of hCG.

5. The aptamer of claim 1, wherein the aptamer is labelled with a label selected from the group consisting of biotin, a fluorescent label, a luminescent label, a radioactive isotope, amine, aryl azides, thiol, a nanoparticle, or an enzymatic label.

6. A biosensor device for detecting human chorionic gonadotropin (hCG), comprising at least one aptamer comprising or consisting of the nucleotide sequence of any one of SEQ ID NOs: 7-9, or a complementary sequence thereof, wherein the aptamer selectively and/or specifically binds hCG.

7. The biosensor device of claim 6, wherein the aptamer has the nucleotide sequence of any one of SEQ ID Nos: 11-13, or a complementary sequence thereof.

8. The biosensor device of claim 6, wherein the biosensor device comprises two aptamers.

9. The biosensor device of claim 8, comprising an aptamer comprising or consisting of the nucleotide sequence of SEQ ID NO: 7 or SEQ ID NO:8, or a complementary sequence thereof, and an aptamer comprising or consisting of the nucleotide sequence of SEQ ID NO:9, or a complementary sequence thereof.

10. The biosensor device of claim 6, wherein the at least one aptamer binds the β subunit of hCG.

11. The biosensor device of claim 10, wherein the aptamer binds the β₁ epitope of hCG.

12. The biosensor device of claim 6, wherein the at least one aptamer is labelled with a label selected from the group consisting of biotin, a fluorescent label, a luminescent label, a radioactive isotope, amine, aryl azides, thiol, a nanoparticle, or an enzymatic label.

13. A method of detecting human chorionic gonadotropin (hCG) in a sample, the method comprising:
a) providing an aptamer having selectivity and/or specificity for binding human chorionic gonadotropin (hCG), wherein the aptamer comprises or consists of the nucleotide sequence of any one of SEQ ID NOs: 7-9, or a complementary sequence thereof;
b) contacting the sample with the aptamer; and
c) detecting binding of the aptamer to hCG, wherein binding of the aptamer to hCG indicates the presence of hCG in the sample.

14. The method of claim 13, wherein the aptamer has the nucleotide sequence of any one of SEQ ID Nos: 11-13, or a complementary sequence thereof.

15. The method of claim 13, wherein the method is a sandwich assay and further comprises contacting the aptamer bound to the hCG with a second aptamer.

16. The method of claim 15, wherein the aptamer bound to the hCG comprises or consists of the nucleotide sequence of SEQ ID NO: 7 or SEQ ID NO:8, or a complementary sequence thereof, and the second aptamer comprises or consists of the nucleotide sequence of SEQ ID NO:9, or a complementary sequence thereof.

17. The method of claim 13, wherein the aptamer binds the β subunit of hCG.

18. The method of claim 17, wherein the aptamer binds the β₁ epitope of hCG.

19. The method of claim 13, wherein the aptamer is labelled with a label selected from the group consisting of biotin, a fluorescent label, a luminescent label, a radioactive isotope, amine, aryl azides, thiol, a nanoparticle, or an enzymatic label.

20. The method of claim 13, wherein detecting binding of the aptamer to hCG is performed using an impedimetric assay, a spectrophotometric assay, a voltammetric assay, a chemiluminescence cytometry assay, a radioactive assay, an immunochromatographic assay, a piezoelectric assay, a colourimetric assay, a fluorescence
